(19) United States
(12) Patent Application Publication
(10) Pub. No.: US 2005/0106086 A1
(43) Pub. Date: May 19, 2005

(54) Movable gas-to-liquid system and process

(76) Inventors: H. Lynn Tomlinson, Tulsa, OK (US); E. Gary Roth, Spring, OK (US); Kenneth Agee, Bixby, OK (US)

Correspondence Address: JENKENS & GILCHRIST, 1401 MCKINNEY, SUITE 2600, HOUSTON, TX, 77010, US

(21) Appl. No.: 10/994,857

(22) Filed: Nov. 22, 2004

Related U.S. Application Data
(63) Continuation-in-part of application No. 10/913,892, filed on Aug. 6, 2004.
(60) Provisional application No. 60/493,293, filed on Aug. 6, 2003.

(51) Int. Cl. C07C 27/26; B01J 10/00

(52) U.S. Cl. 422/187; 518/726

(57) ABSTRACT

A system having a movable platform including synthesis gas production, synthetic crude production and product upgrading is provided. The system may include one or more movable platforms on which the various production and/or upgrading facilities are located. A process for converting natural gas to hydrocarbon products is also provided where the process occurs on a movable platform. The process may occur on one or more operationally connected vessels. The movable platform may be any of a number of movable or transportable bases on which process equipment may be placed and/or in which hydrocarbon products may be stored.

### CROSS REFERENCE TO RELATED APPLICATIONS

This application is a continuation-in-part application of U.S. Ser. No. 10/913,892, filed on Aug. 6, 2004 which claimed priority to U.S. provisional application Ser. No. 60/493,293, filed on Aug. 6, 2003, the disclosure of which is incorporated herein by reference.

### FEDERALLY SPONSORED RESEARCH

Not applicable.

### REFERENCE TO MICROFICHE APPENDIX

Not applicable.

### FIELD OF THE INVENTION

The invention relates to a movable gas-to-liquid system and process, and more particularly, to a gas-to-liquid system constructed on a marine vessel, such as an FPSO.

### BACKGROUND OF THE INVENTION

Fischer-Tropsch processes for converting synthesis gas into higher carbon number hydrocarbons are well known. The hydrocarbon products of a Fischer-Tropsch synthesis generally include a wide range of carbon number, ranging from between about 1 and about 100. The end products which may be recovered from the Fischer-Tropsch synthesis product, following separation, hydroprocessing or other upgrading, include but are not limited to liquified petroleum gas (“LPG”), naphtha, middle distillate fuels, e.g. jet and diesel fuels, and lubricant basestocks. Some of these end products, however, are more desirable than others for a variety of reasons, including for example, being marketable at a higher margin.

The desirability of an end product of a Fischer-Tropsch synthesis may also be dependent upon geographic location of the Fischer-Tropsch plant.

While technological advances within the energy industry have made dramatic improvements in lowering the cost of finding, producing and refining oil, vast quantities of remote and stranded gas still wait to be developed. Gas to liquid (“GTL”) technologies may assist in developing and monetizing these resources. Such GTL technologies are especially critical to offshore applications given that about one-half of the world's stranded gas is located within submerged formations.

In conventional GTL processes, synthesis gas is generated from natural gas via partial oxidation with oxygen, requiring an air separation plant to provide the oxygen. In conventional approaches, nitrogen is eliminated from the synthesis gas stream as an unwanted inert. In an air-based system, however, synthesis gas is produced by oxidation of hydrocarbons using air- or oxygen enriched air-carried oxygen, rather than separated oxygen. This eliminates the expense, as well as the extra space requirment, of an air separation plant. It thus reduces capital costs, making possible plants with considerably smaller footprints, and also provides for a safer operating environment.

Fischer-Tropsch plants of at least about 50,000 B/d production are generally required in order to lower the capital cost per barrel of daily capacity to an acceptable level. However, such Fischer-Tropsch plants require about 500 Mmcf/d of feed gas, or 5.4 trillion cubic feet over a thirty year period. Only about 2% of the known gas fields outside of North America are of such size.

Stranded natural gas reserves also may produce condensates and liquified petroleum gasses (LPGs), i.e. propanes and butanes, which may be recovered. Isolation of LPG components, with or without combination with Fischer-Tropsch produced LPGs, is not typically practiced in gas to liquid processes. However, failure to monetize LPG components further lowers the economic feasibility of accessing and producing stranded gas reserves.

There remains a need therefore, for a process for converting stranded gas reserves having a capacity less than 5.4 trillion cubic feet, and preferably having between about 0.5 and 5.0 trillion cubic feet natural gas, efficiently and economically into higher value hydrocarbon products. There remains a further need for a gas to liquid process monetizing LPG components recovered from stranded gas reserves as well as those LPG components produced in Fischer Tropsch processes. There remains a further need for a process which may be transported one or more times to natural gas reserve locations. There is a further need for a modularized system which may be configured and re-configured to produce a product slate adapted to meet market and local needs and conditions.

### SUMMARY OF THE INVENTION

The invention provides a movable gas to liquids system and process. In some embodiments of the invention, a synthesis gas production unit, a synthetic crude production unit and a product upgrading unit are located on a movable platform wherein the units are operationally connected to each other.

In another embodiment of the invention, a process for converting natural gas to hydrocarbon liquids is provided wherein the process occurs on one or more movable platforms operationally connected to each other.

### BRIEF DESCRIPTION OF THE DRAWINGS

FIG. 1 is a schematic drawing of a GTL FPSO embodiment of the present invention.

FIG. 2 is a schematic drawing of an Oil/GTL FPSO embodiment of the present invention.

FIG. 3 is a schematic diagram showing an alternative embodiment of the invention wherein Wellhead Natural Gas and Fischer-Tropsch synthesis product are blended in the production of LPG products, Naphtha products and transportation fuel products.

FIG. 4 is a schematic diagram showing an alternative embodiment of the invention wherein Wellhead Natural Gas, Fischer-Tropsch synthesis product, and imported offsite Natural Gas are co-processed in the production of a transportation fuel.

### DESCRIPTION OF EMBODIMENTS OF THE INVENTION

The term “Cₓ” where x is a number greater than zero, refers to a hydrocarbon compound having predominantly a carbon number of x. As used herein, the term Cₓ may be modified by reference to a particular species of hydrocarbons, such as, for example, C₅ olefins. In such instance, the term means an olefin stream comprised predominantly of pentenes but which may have impurity amounts, i.e. less than about 10%, of olefins having other carbon numbers such as hexene, heptene, propene, or butene. Similarly, the term “Cₓ+” refers to a stream wherein the hydrocarbons are predominantly those having a hydrocarbon number of x or greater but which may also contain impurity levels of hydrocarbons having a carbon number of less than x. For example, the term C₁₅₊ means hydrocarbons having a carbon number of 15 or greater but which may contain impurity levels of hydrocarbons having carbon numbers of less than 15. The term “Cₓ-C_{y}”, where x and y are numbers greater than zero, refers to a mixture of hydrocarbon compounds wherein the predominant component hydrocarbons, collectively about 90% or greater by weight, have carbon numbers between x and y. For example, the term C₅-C₉ hydrocarbons means a mixture of hydrocarbon compounds which is predominantly comprised of hydrocarbons having carbon numbers between 5 and 9 but may also include impurity level quantities of hydrocarbons having other carbon numbers.

Unless otherwise specified, all quantities, percentages and ratios herein are by weight.

Synthesis gas (or “syngas”) useful in producing a Fischer-Tropsch product useful in the invention may contain gaseous hydrocarbons, hydrogen, carbon monoxide and nitrogen with H₂:CO ratios from between about 0.8:1 to about 3.0:1. The hydrocarbon products derived from the Fischer-Tropsch reaction may range from methane to high molecular weight paraffinic waxes containing more than 100 carbon atoms. Operating conditions and parameters of an autothermal reactor for producing a syngas useful in the process of the invention are well known to those skilled in the art. Such operating conditions and parameters include but are not limited to those disclosed in U.S. Pat. Nos. 4,833,170; 4,973,453; 6,085,512; 6,155,039, the disclosures of which are incorporated herein by reference.

Fischer-Tropsch catalysts are also known in the art and include, those based upon for example, cobalt, iron, ruthenium as well as other Group VIIIB transition metals or combinations of such metals, to prepare both saturated and unsaturated hydrocarbons. The Fischer-Tropsch catalyst may also include a support, such as a metal-oxide support, including but not limited to silica, alumina, silica-alumina or titanium oxides. For example, a cobalt (Co) catalyst on transition alumina with a surface area of approximately 100-200 m²/g may be used in the form of spheres of 50-150 μm in diameter. The Co concentration on the support may be between about 5 wt % to about 30 wt %. Certain catalyst promoters and stabilizers may be used. The stabilizers include Group IIA or Group IIIB metals, while the promoters may include elements from Group VIII or Group VIIB. The Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidizing bed, or continuously stirred tank reactor (“CSTR”). The FTR may be operated at a pressure from about 100 psia to about 800 psia and a temperature from about 300° F. to about 600° F. The reactor gas hourly space velocity (“GHSV”) may be from about 1000 hr⁻¹ to about 15000 hr⁻¹. Operating conditions and parameters of the FTR useful in the process of the invention are well known to those skilled in the art. Such operating conditions and parameters include but are not limited to those disclosed in U.S. Pat. Nos. 4,973,453; 6,172,124; 6,169,120; and 6,130,259, the disclosures of which are incorporated herein by reference.

Some embodiments of the invention provide a movable system optimized for the monetization of stranded gas reserves. In preferred embodiments of the invention, the stranded gas reserves are located in or near submerged formations, such as those found off-shore. The movable system may be moved, for example, by way of ocean- or sea-going vessels, such as a floating production, storage and offloading (FPSO) vessel. Movable vessels useful in the invention may be independently mobile or may require external mobility means, such as lift ship or tugboat. As used herein, the terms movable platforms and/or vessels include, without limitation, FPSOs, floating storage and offloading vessels (FSO), gravity based structures, spar platforms, tension leg platforms. However, other movable platforms are included in the scope of the invention, including trailer, truckbed, rail car or platform, or other movable forms on which the modules may be transported or moved from location to location. In some embodiments of the invention, the movable platform is maintained in place by any of a number of methods, including without limitation, fixed turret, removable turret, conventional mooring systems, anchoring, and/or suction piles.

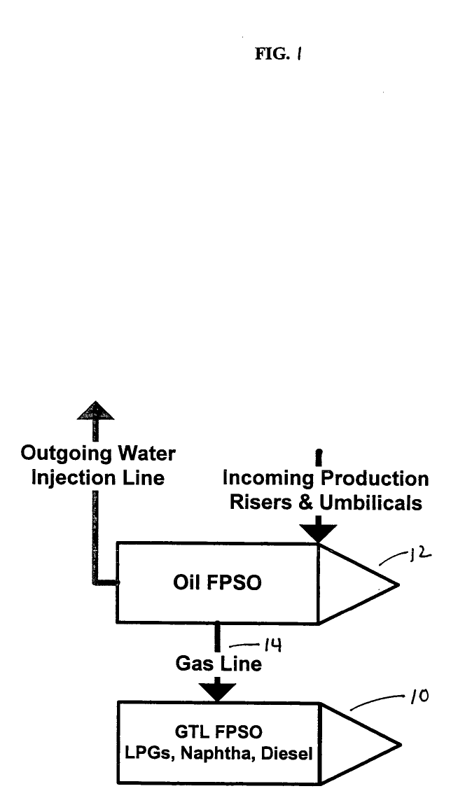

Referring to FIG. 1, one embodiment of the invention is shown in which a GTL FPSO 10 is located in a position accessible to an existing FPSO 12, generally accessible by pipeline. Existing FPSO 12 may be an oil and or gas producing FPSO or any other type of FPSO from which natural gas may be obtained. In some embodiments of the invention, existing FPSO 12 is a crude oil production FPSO from which associated natural gas may be obtained. Existing FPSO 12 is, in some embodiments, an FPSO which is in place and producing prior to the introduction of the GTL FPSO 10. In other embodiments, existing FPSO 12 and GTL FPSO 10 are placed in proximity at or substantially at the same time. The GTL FPSO 10 receives natural gas from existing FPSO 12 through gas pipeline 14. Systems located on the GTL FPSO 10 convert the natural gas into a synthesis gas, and the synthesis gas into a synthetic crude using known processes. In some embodiments of the invention, the GTL FPSO 10 further includes product upgrading and recovery facilities for the conversion of synthetic crude into one or more products, such as naphtha and transportation fuels, including, for example, diesel fuel. As used herein, the term “product upgrading” means the refining of a synthetic crude that is waxy, into one or more hydrocarbon products, including for example, a single wide-boiling range product (e.g., C5 to C40) having a reduced pour point which is lower than the waxy synthetic crude which is sufficient to prevent wax crystallization during transshipment either as a separate product or blended with crude oil and/or condensate, naphthas, liquified petroleum gases, basestocks, solvents, kerosene, and hydrocarbon products meeting fuel specifications. In some embodiments, product upgrading eliminates all or all but trace amounts of oxygenated compounds. In some embodiments, the addition of some additives to the product upgrading products of kerosene and fuels may be required before end use. Similarly, any of the products may, in some embodiments, be blended with petroleum products prior to end use. As used herein, the term reduced pour point means having a pour point between about 100° F. and about 0° F., between about 70° F. and about 20° F., between about 50° F. and about 20° F., or between about 40° F. and about 20° F.

On the GTL FPSO 10 is located a syngas production unit which may include those components and may be of a type known in the art. Similarly, a synthetic crude production unit of a type and including components known to those in the art is also located on the GTL FPSO 10. In some embodiments, product upgrading units including components known in the art, such as hydrocrackers, distillation columns, dehydration and oligomerization reactors, are located on the GTL FPSO 10. As used herein, the term “product upgrading” refers to the production of finished end user products, such as diesel fuel, and/or intermediate products, such as lubricant basestocks.

In some embodiments of the invention as described in FIG. 1, the synthesis gas and synthetic crude production units and, if present, the product upgrading units are mounted onto the GTL FPSO in a manner which causes such units to be substantially unmovable. In alternative embodiments of the invention, some or all of such units are mounted on skids or modules which may be interchanged and/or repositioned.

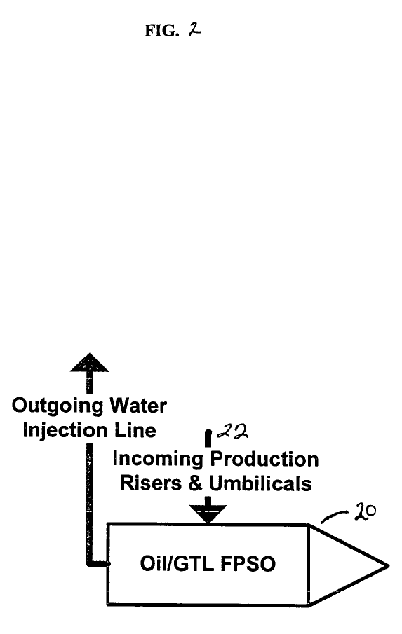

Referring to FIG. 2, an embodiment of the invention is shown in which an Oil/GTL FPSO 20 is located at or near an oil or natural gas reserve. In the case of an oil reserve, the Oil/GTL FPSO 20 preferably includes oil production facilities as well as the GTL component units as discussed in connection with FIG. 1. In the case of a natural gas reserve, the Oil/GTL FPSO 20 includes those GTL components, i.e., synthesis gas production unit, Fischer-Tropsch unit, as discussed in connection with FIG. 1. The Oil/GTL FPSO includes primary separation equipment to separate oil, gas and water which may all, or some, be produced from the wellhead. As shown in FIG. 2, the Oil/GTL FPSO 20 communicates with the wellhead through incoming production risers and umbilicals 22 and one or more outgoing water injection lines 24.

In some embodiments of the invention as described in FIG. 2, the synthesis gas and synthetic crude production units and, if present, the product upgrading units are mounted onto the GTL FPSO in a manner which causes such units to be substantially unmovable. In alternative embodiments of the invention, some or all of such units are mounted on skids or modules which may be interchanged and/or repositioned.

In preferred embodiments of the invention, the GTL FPSO 10 and the Oil/GTL FPSO 20 are configured to produce complete parcels, as that term is commonly used in the shipping field. That is, in preferred embodiments of invention, the storage capacity of the FPSO and the production capacity of oil production and/or gas to liquid production facilities are correlated so as to completely or nearly completely fill the storage capacity of the FPSO. Further, the storage tanks on the GTL FPSO are preferably sized to match as large a shipping parcel as possible. Shipping of product is a key financial consideration in any project that depends upon product reaching the final market in order to be profitable. In the hydrocarbon product market, shipping of products occurs in clean product tankers, typically. For instance, clean product tankers in the size range of 30,000 dead weight tonnes to 60,000 dead weight tonnes are very common. “Dead weight tonnes” or “dwt” refers to product, ship stores, ship fuel, and consumables on-board a ship and the dwt rating of a ship is often used to correlate the capacity in barrels of the ship. Typically, the staffing of a 30,000 dwt ship versus a 60,000 dwt ship are the same. Also, there is typically little difference in the speed at which a 30,000 dwt ship versus a 60,000 dwt ship can travel. There then remains two main variables for a product owner to reduce shipping cost; 1) reduce distance of travel and 2) increase the parcel size. Therefore, shipping parcels that are as large as possible tend to set the size of the storage at the production site. The integration of a GTL FPSO onto an existing crude oil tanker or onto a new hull the size of a crude oil tanker is driven by two variables which are i) the deck space required for the topside equipment required for processing the wellhead stream(s) and ii) the storage requirements of the produced liquids. In the case of the Oil/GTL FPSO, there are 4 types of products: 1) crude oil and/or condensate; 2) LPG; 3) Naphtha; and 4) middle distillate fuel. In another embodiment of the invention, base oil product is also stored. The storage for the products is dictated by the shipping parcel size and the relative production ratios of the products. For example, if an Oil/GTL FPSO is installed on an oil field and the associated gas contains a tremendous amount of LPG-type materials, then the optimum LPG storage requirement may decrease the crude oil storage component. Table 1 shows three common crude oil tanker hull sizes. In Table 1, the common shipping parcel sizes were applied and the resultant storage on the FPSO is shown. Note that LPG's are typically shipped by volume rather than weight. Currently available FPSOs have capacities ranging between about 200,000 barrels to about 2.4 million barrels. However, the invention contemplates FPSOs having larger and smaller storage capacities.

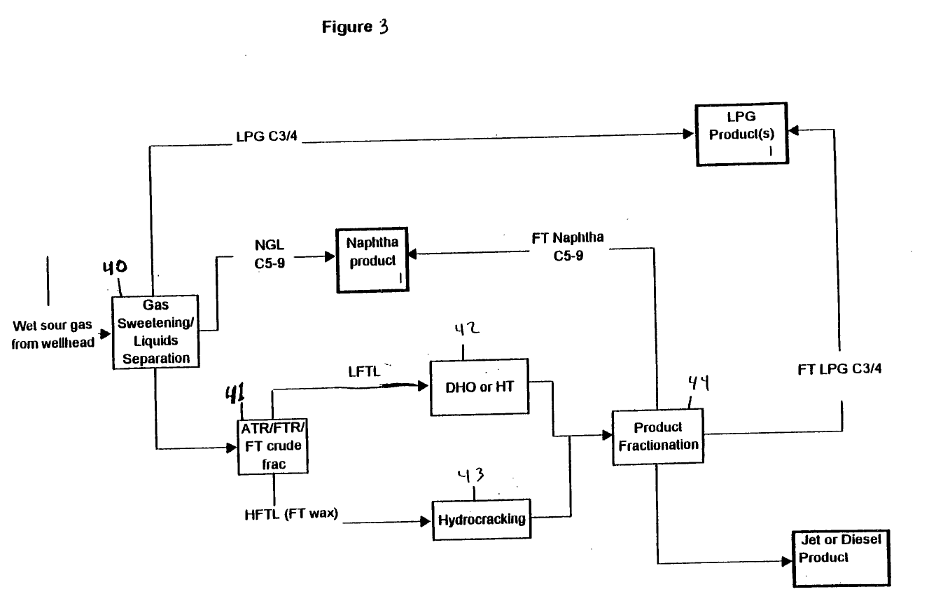

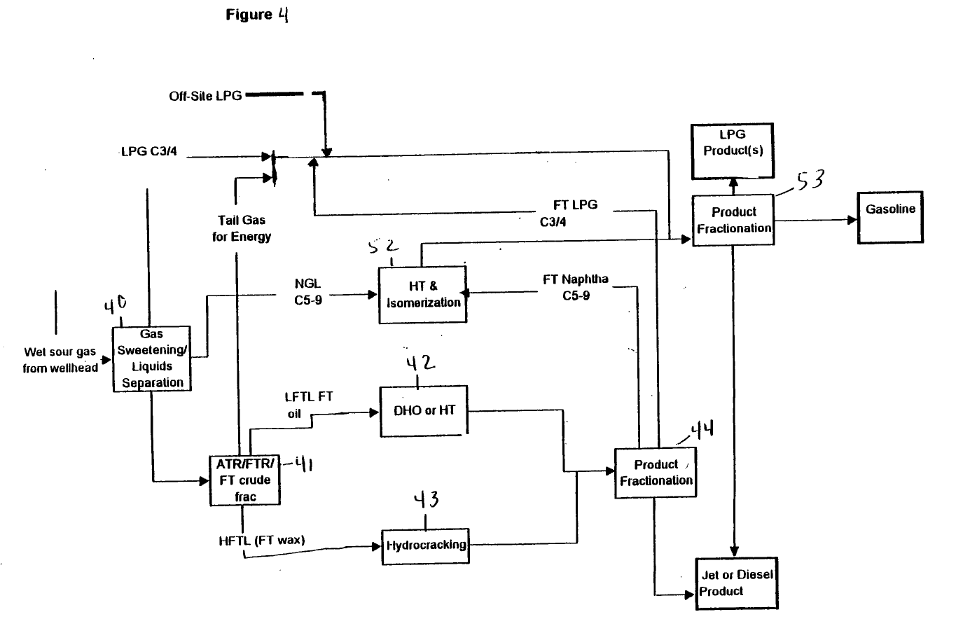

Some embodiments of the invention further provide for cooled storage of LPG products, the production of which is discussed in more detail below in connection with FIGS. 3 and 4. Such cooled storage facility is preferably correlated with production capacity of LPG and LPG components so as to achieve complete parcel shipping amounts.

While it is current practice to store LPG and LPG components at cooled temperatures so as to maintain such products liquefied, the invention contemplates the use of both or either temperature and pressure to maintain such products in a liquid state.

Although FIGS. 1 and 2 describe the use of an FPSO vessel as the movable platform for the production and GTL facilities, other embodiments of the invention include other land and marine based platforms, such as ships, gravity based structures, and platforms.

In yet other embodiments of the invention, the movable platform may have a multi-vessel configuration. Multi-vessel configurations permit the use of any of the component vessels to be used for a single or multiple purposes. For example, in a two-vessel configuration, one of the two vessels may be used, for example, for oil production equipment, primary separation, and crude oil storage. The second vessel could be employed, for example, for synthesis gas and synthetic crude production along with storage of LPG components, i.e., butane and propane, LPG, end user products, and/or intermediate products. In such an embodiment, both vessels would preferably be FPSOs. In an alternative three vessel configuration, one vessel could function for all production activities, including for example, oil production, oil/gas separation, LPG recovery, synthesis gas and synthetic crude production. In such alternative structure, the other two vessels could be floating storage and offloading vessels (FSOs). In some such three vessel embodiments, the FSOs may be such so as to allow cold and or warmed storage. Yet other compartments of the FSOs could be temperature untreated. In some embodiments, end user and/or intermediate GTL products could be stored in appropriate tanks located on either of the FSO vessels. In alternative embodiments, one of the vessels may be dedicated to crude oil storage and one of the vessels dedicated to end user, intermediate, LPG, or LPG component storage.

Referring to FIG. 3, one embodiment of the invention is shown in which wet sour gas from a wellhead is processed in a gas sweetening/liquids separation unit 40. An LPG fraction is obtained overhead from unit 40 (“NG-LPG” hereinafter), a natural gas liquids (“NGL” hereinafter) fraction is obtained as an upper side stream from unit 40 containing primarily hydrocarbons having a carbon number greater than 5 is recovered. The bottoms fraction, known in the art as “residue gas” containing primarily hydrocarbons having a carbon number equal to or greater than 9 is recovered. The bottoms fraction is sent to synthesis gas production. The synthesis gas so produced may then be used in a GTL production, such as a Fischer-Tropsch synthesis. The Fischer-Tropsch synthesis product may then be fractionated to recover a light Fischer-Tropsch liquid, also commonly referred to as a Fischer-Tropsch oil, (“LFTL”) and a heavy Fischer-Tropsch liquid, also commonly referred to as a Fischer-Tropsch wax, (“HFTL”). In FIG. 3, the synthesis gas production, Fischer-Tropsch synthesis and Fischer-Tropsch product fractionation processes are jointly illustrated in unit 41. The Fischer-Tropsch LFTL may then be dehydrated, or otherwise treated for removal of oxygenates, or hydrotreated, as indicated in FIG. 4 in unit 42. The HFTL may be hydrocracked in unit 43 to produce lower molecular weight hydrocarbons. The products of units 42 and 43 may then be recombined and fed into a product fractionator 44. A Fischer-Tropsch LPG product (“FT-LPG” or “GTL-LPG” hereinafter)) may be recovered from Fractionator 44 and combined with the LPG obtained overhead from unit 40. The combined LPG may then be further processed into a variety of known LPG products. For example, any of the FT-LPG product, NG-LPG, or a combination of FT-LPG and NG-LPG products may be separated into butane and propane fractions. A Fischer-Tropsch or GTL Naphtha may also be recovered from fractionator 44 and combined with the NGL recovered from unit 40. The combined NGL and Fischer-Tropsch Naphtha may then be further processed into a variety of known naphtha products. Transportation fuels, or blending stocks therefor, such as jet fuel and diesel fuel may also be recovered from fractionator 44.

FIG. 4 illustrates yet another embodiment of the invention in which liquified petroleum gas is imported from an off-site location and co-processed with the LPG fraction recovered from unit 40 and the Fischer-Tropsch LPG fraction recovered from fractionator 44. The embodiment of the invention shown in FIG. 4 could be used with stranded reserves providing even less than 100 Mmcf/d but which may provide feasible economics due to the ability to supplement the stranded gas reserve with imported natural gas.

In another embodiment of the invention, the processes depicted in the foregoing embodiments are modularized such that a single processing plant may be alternately configured to process various components of a stranded gas stream as well as to alternately process such stream into various products and product slates. For example, a synthesis gas module may include a gas sweetening/liquids separation unit for removal of certain contaminants, such as sulfur, and separation of liquids from gaseous hydrocarbon components. Such synthesis gas module would generally also include an autothermal reactor for conversion of the gaseous hydrocarbons into synthesis gas. The synthesis gas module may also include one or more Fischer-Tropsch reactors. Alternatively, the Fischer-Tropsch reactor(s) may be combined with one or more Fischer-Tropsch product fractionators to form a Fischer-Tropsch module. One or more product modules may be connected to the synthesis gas module or Fischer-Tropsch module for upgrading the product of the Fischer-Tropsch synthesis into one or more higher value products. One example product module, a transportation fuel product module, would include dehydration or hydrotreatment units for the processing of an LFTL fraction as well as a hydrocracking unit for the processing of an HFTL fraction to obtain a synthetic transportation fuel. Other product modules include, for example, a hydrotreatment plus hydroisomerization unit and a dehydrogenation plus oligomerization units. In some embodiments of the invention, off-site or imported natural gas feed may be piped directly into a product unit.

Embodiments of the invention provide one or more of the following advantages:
(1) economically feasible recovery of stranded natural gas reserves;
(2) movable system format permitting transportation to and operation at or near the location of the natural gas reserve;
(3) modular plant design permitting product slate adaptation; and
(4) any of (1)-(3) above incorporating storage in the hull of the movable platform which has been optimized to accommodate optimum parcel sizes.

The following examples illustrate embodiments of the invention but are not intended to limit the scope of the invention.

### EXAMPLE 1

As discussed in detail above, Table 1 shows three possible arrangements of the invention using standard crude oil tanker hulls as a base hull for the Oil/FPSO and/or GTL FPSO.

| | | | |
|---|---|---|---|
| Product | Minimum | Medium | Optimum |

Shipping Matrix - in Shipping Units

| | | | |
|---|---|---|---|
| Crude Oil | 80,000 DWT | 115,000 DWT | 150,000 DWT |
| | Aframax | Aframax | Suezmax |
| Propane | 35,000 M3 | 55,000 M3 | 75,000 M3 |
| Butane | 35,000 M3 | 55,000 M3 | 75,000 M3 |
| Naphtha | 25,000 DWT | 55,000 DWT | 85,000 DWT |
| Diesel | 25,000 DWT | 55,000 DWT | 85,000 DWT |

FPSO/FSO Storage Requirements - in Barrels

| | | | |
|---|---|---|---|
| Crude Oil | 592,000 | 851,000 | 1,110,000 |
| Propane | 220,150 | 345,950 | 471,750 |
| Butane | 220,150 | 345,950 | 471,750 |
| Naphtha | 221,479 | 487,254 | 753,028 |
| Diesel | 204,221 | 449,286 | 694,351 |
| Total | 1,458,000 | 2,479,439 | 3,500,879 |
| Crude % | 41% | 34% | 32% |

### EXAMPLE 2

Table 2 shows an alternate embodiment of the invention whereby a new or existing FPSO associated with crude oil production is connected to a GTL FPSO.

| | | Dual Ship | |
|---|---|---|---|
| Product | Single Ship | Crude Oil FPSO | GTL FPSO |

Shipping Matrix - in Shipping Units

| | | | |
|---|---|---|---|
| Crude Oil | 115,000 DWT | As Per Client | |
| | Aframax | | |
| Propane | 75,000 M3 | | 75,000 M³ |
| Butane | 55,000 M3 | | 55,000 M³ |
| Naphtha | 25,000 DWT | | 25,000 DWT |
| Diesel | 55,000 DWT | | 85,000 DWT |

Shipping Matrix - in Barrels

| | | | |
|---|---|---|---|
| Crude Oil | 851,000 | As Per Client | 0 |
| Propane | 345,950 | 0 | 471,750 |
| Butane | 157,250 | 0 | 345,950 |
| Naphtha | 221,479 | 0 | 221,479 |
| Diesel | 449,286 | 0 | 449,286 |
| Totals | 2,024,965 | As Per Client | 1,488,465 |

1. A system for converting natural gas into one or more liquid hydrocarbon products comprising:
(a) a movable platform on which synthesis gas production, synthetic crude production, and product upgrade units are located, wherein the synthesis gas production unit, the synthetic crude production unit, and the product upgrade unit are operationally connected.

2. The system of claim 1 wherein the movable platform is a floating production, storage, and offloading (FPSO) vessel.

3. The system of claim 1 wherein the product upgrade unit comprises an LPG component separation column.

4. The system of claim 1 wherein the system does not include an air separation facility.

5. The system of claim 4 wherein the synthesis gas production unit utilizes air or oxygen-enriched air.

6. The system of claim 1 wherein the synthetic crude production unit comprises a Fischer-Tropsch reactor.

7. The system of claim 1 wherein the synthesis gas production unit comprises an autothermal reactor.

8. The system of claim 1 wherein the movable platform is transported by means selected from the group of tugboat, barge and heavy lift ship.

9. The system of claim 2 wherein the FPSO comprises one or more storage tanks selected from the group of cooled tanks, warmed tanks, and temperature untreated tanks.

10. The system of claim 9 wherein the cold storage tanks are maintainable at temperatures and pressures sufficient to store LPG or LPG components.

11. The system of claim 1 wherein the product upgrading unit comprises a subsystem for upgrading synthetic crude into a transportation fuel.

12. The system of claim 11 wherein the transportation fuel is diesel fuel.

13. The system of claim 9 wherein the cold storage tanks of the FPSO have a total capacity of about 820,000 barrels.

14. The system of claim 15 wherein the total storage capacity of the FPSO is about 1.6 million barrels.

15. The system of claim 2 wherein the total storage capacity of the FPSO is between about 1.4 and 3.6 million barrels.

16. The system of claim 15 wherein the FPSO comprises cold storage tanks and wherein the combined capacity of the cold storage tanks is between about 440,000 and about 1 million barrels.

17. The system of claim 1 further comprising a gas sweetening/liquids separation unit for receiving a wet sour gas and recovering an LPG fraction.

18. The system of claim 1 further comprising an oil production unit and an oil/gas separation unit.

19. The system of claim 18 wherein the movable platform is an FPSO.

20. The system of claim 19 wherein the FPSO comprises cold storage tanks and hot storage tanks.

21. The system of claim 20 wherein the cold storage tanks are maintainable at temperatures and pressures sufficient to store LPG or LPG components.

22. The system of claim 21 wherein the combined cold storage tank capacity is about 500,000 barrels.

23. The system of claim 22 wherein the total storage capacity of the FPSO is between about 2 and 2.2 million barrels.

24. A gas-to-liquids process comprising the steps of:
(a) transporting a movable platform comprising a synthesis gas production unit, a synthetic crude production unit, and a product upgrading unit to a location at or near a natural gas containing reserve;
(b) receiving a natural gas stream from the reserve and converting the natural gas stream into a synthesis gas in the synthesis gas production unit;
(c) converting the synthesis gas into synthetic crude in the synthetic crude production unit;
(d) converting at least a portion of the synthetic crude into a finished end-user or intermediate product in the product upgrading unit.

25. The process of claim 24 wherein the movable platform does not include an air separation facility.

26. The process of claim 24 wherein air or oxygen enriched air is used in the synthesis gas production unit.

27. The process of claim 24 further comprising the step of recovering a LPG fraction from the synthetic crude.

28. The process of claim 24 wherein the movable platform further comprises a gas sweetening/liquids separation unit and further comprising the steps of recovering a LPG fraction from the natural gas stream.

29. The process of claim 24 wherein the movable platform further comprises a gas sweetening/liquids separation unit and further comprising the steps of recovering a NG-LPG fraction from the natural gas stream and recovering a GTL-LPG fraction from the synthetic crude.

30. The process of claim 29 further comprising the step of storing one or both of the NG-LPG and GTL-LPG in cold storage at temperatures and pressures sufficient to maintain the one or both of the NG-LPG and GTL-LPG liquefied.

31. The process of claim 24 wherein the step of converting at least a portion of the synthetic crude into a finished end-user or intermediate product in the product upgrading unit comprises converting at least a portion of the synthetic crude into a transportation fuel.

32. The process of claim 31 wherein the transportation fuel comprises diesel fuel.

33. The process claim 24 wherein the step of converting at least a portion of the synthetic crude into a finished end-user or intermediate product in the product upgrading unit comprises production of a complete parcel of combined liquid products.

34. The process of claim 33 further comprising the steps of offloading and shipping the complete parcel of combined liquid products from the movable platform.

35. The process of claim 24 wherein the natural gas containing reserve is a stranded natural gas reserve.

36. The process of claim 24 wherein the natural gas containing reserve is an oil reserve which produces associated natural gas.

37. The process of claim 36 wherein the movable platform further comprises an oil production unit and an oil/gas separation unit.

38. The process of claim 36 wherein the step of receiving a natural gas stream from the reserve comprises the steps of separating the associated natural gas from produced oil on an Oil FPSO and transporting the separated associated natural gas from the Oil FPSO to the movable platform through of a gas line.
